(19) United States
(12) Patent Application Publication
(10) Pub. No.: US 2026/0083822 A1
(43) Pub. Date: Mar. 26, 2026

(54) Topical Ocular Insulin Delivery Enhanced by Prostaglandin Analogues

(71) Applicant: David Harold Gosiengfiao, Taguig City (PH)

(72) Inventor: David Harold Gosiengfiao, Taguig City (PH)

(21) Appl. No.: 19/403,706

(22) Filed: Nov. 28, 2025

Related U.S. Application Data
(63) Continuation-in-part of application No. PCT/PH2024/050008, filed on Jun. 13, 2024.
(63) Continuation-in-part of application No. PCT/IB2025/058784, filed on Sep. 1, 2025.

(30) Foreign Application Priority Data
Jun. 13, 2024 (WO) PCT/PH2024/050008
Jun. 13, 2024 (WO) PCT/PH2024/050008
Sep. 1, 2025 (WO) PCT/IB2025/058784

(51) Int. Cl. A61K 38/28; A61K 9/00; A61K 31/5575; A61P 3/08

(52) U.S. Cl. CPC A61K 38/28; A61K 9/0048; A61K 31/5575; A61P 3/08

(57) ABSTRACT

A pharmaceutical composition is disclosed comprising an insulin, a set of excipients, and a penetration enhancer, wherein the penetration enhancer is a prostaglandin analogue configured to enhance the permeability of insulin upon topical ocular administration. The composition enables increased transport of insulin across ocular tissues, improving absorption and bioavailability following topical delivery to the eye. The formulation includes pharmaceutically acceptable excipients such as surfactants, buffering agents, viscosity enhancers, tonicity agents, preservatives, and solvents suitable for ophthalmic use. The invention provides a non-invasive route of administering insulin through the eye, allowing systemic or local therapeutic effects without injection.

### CROSS REFERENCE TO RELATED APPLICATIONS

This application is filed as a continuation-in-part (CIP) of International Patent Application No. PCT/PH2024/050008, filed on Jun. 13, 2024, and International Patent Application No. PCT/IB2025/058784, filed on Sep. 1, 2025, each of which is hereby incorporated by reference in its entirety for all purposes. The present application claims the benefit of priority to each of the foregoing applications to the fullest extent permitted under applicable law.

### TECHNICAL FIELD OF THE INVENTION

This invention relates to a method of increasing the systemic hypoglycemic effect of insulin when applied topically to the eye through the use of prostaglandin analogues as penetration enhancers, more specifically, to an ophthalmic composition of insulin and prostaglandin analogue for the treatment of diabetes mellitus.

### BACKGROUND OF THE INVENTION

Diabetes mellitus is a chronic metabolic disorder characterized by hyperglycemia or elevated blood sugar levels, affecting millions of individuals worldwide (International Diabetes Federation, 2019).

According to the International Diabetes Federation (IDF), as of 2021, an estimated 537 million adults worldwide are living with diabetes, which represents approximately one in eleven adults. Due to factors such as aging populations, urbanization, and lifestyle changes, including unhealthy diets and reduced physical activity, the prevalence of diabetes is increasing globally. Diabetes is a significant cause of morbidity and mortality, with complications such as cardiovascular disease, kidney disease, nerve damage, and blindness. The IDF estimates that diabetes was responsible for 4.2 million deaths in 2019, making it one of the leading causes of death worldwide.

Insulin therapy is the cornerstone of treatment for type 1 diabetes and often required for advanced type 2 diabetes.

Insulin is a hormone used to lower blood glucose to normal levels. It achieves its hypoglycemic effect by promoting the uptake and storage of glucose in the body's tissues. There are several types of insulin products available, which can be classified based on their onset, peak, and duration of action. These differences in action profiles allow healthcare providers to tailor insulin therapy to the individual needs of patients with diabetes. The main types of insulin include:

### Rapid-Acting Insulin:

These insulins start working within 15 minutes of administration, peak at approximately 1 hour, and last for 2-4 hours. They are typically taken just before or with meals to help control post-meal blood sugar spikes. Examples include insulin lispro (Humalog), insulin aspart (NovoLog), and insulin glulisine (Apidra).

### Short-Acting Insulin:

Also known as regular or soluble insulin, short-acting insulin begins to work within 30 minutes to an hour, peaks at 2-4 hours, and lasts for 5-8 hours. It is usually taken 30 minutes before meals. Examples include Humulin R and Novolin R.

### Intermediate-Acting Insulin:

Intermediate-acting insulins have a slower onset and longer duration of action compared to short-acting insulin. They start working within 1-2 hours, peak at 4-12 hours, and last for 12-18 hours. They are typically used to provide basal insulin coverage throughout the day. Examples include NPH insulin (Neutral Protamine Hagedorn), such as Humulin N and Novolin N.

### Long-Acting Insulin:

Long-acting insulins provide a slow, steady release of insulin with no pronounced peak, lasting for 15 up to 24 hours or longer. They are used to provide basal insulin coverage and are often combined with rapid- or short-acting insulin to cover mealtime blood sugar spikes. Examples include insulin glargine (Lantus, Toujeo), insulin detemir (Levemir), and insulin degludec (Tresiba).

The chemical properties of different insulin types, including their amino acid sequences and molecular structures, play a crucial role in determining their rate of absorption.

Insulin molecules exist as monomers or can aggregate into dimers or hexamers. These aggregation states influence the rate of insulin absorption. Insulin monomers are more soluble and are absorbed more rapidly than dimers or hexamers.

Below is an overview of the chemical properties of various insulin types and how they affect the rate of absorption:

### Rapid-Acting Insulin Analogs:

Rapid-acting insulins are engineered to have minor alterations in their amino acid sequences compared to human insulin. These changes result in altered insulin molecules that do not self-aggregate into hexamers as readily as regular insulin, promoting faster absorption into the bloodstream.

For example:

Insulin lispro (Humalog) has a reversal of proline and lysine at positions B28 and B29. This change disrupts the hexamer formation and enhances its solubility, leading to faster absorption.

Insulin aspart (NovoLog) has a proline at position B28 replaced with an aspartic acid, which also reduces hexamer formation and increases the rate of absorption.

Insulin glulisine (Apidra) has asparagine at position B3 replaced with a lysine and a lysine at position B29 replaced with a glutamic acid, which results in faster dissociation into monomers and quicker absorption.

### Short-Acting Insulin (Regular Insulin):

Regular insulin, which is structurally identical to human insulin, forms hexamers in the presence of zinc ions. These hexamers must dissociate into dimers and then monomers before they can be absorbed into the bloodstream. This dissociation process slows down the absorption of regular insulin compared to rapid-acting analogs.

### Intermediate-Acting Insulin (NPH):

NPH insulin is a suspension of human insulin combined with protamine, a protein that forms a complex with insulin. The protamine-insulin complex has reduced solubility, which slows down the absorption of insulin from the injection site into the bloodstream, resulting in an intermediate duration of action.

### Long-Acting Insulin Analogs:

Long-acting insulin analogs have modifications in their amino acid sequences or additional components that slow down their absorption and prolong their action.

### For Example:

Insulin glargine (Lantus, Toujeo) has a glycine substituted for asparagine at position A21 and two arginines added to the B-chain. These changes make insulin glargine less soluble at physiological pH, causing it to precipitate at the injection site. The precipitate slowly dissolves over time, providing a slow, steady release of insulin.

Insulin detemir (Levemir) has a myristic acid fatty acid chain attached to lysine at position B29. This modification increases the binding of insulin detemir to albumin in the bloodstream and at the injection site, slowing down its absorption and prolonging its duration of action.

Insulin degludec (Tresiba) has a hexadecanedioic acid side chain at position B29, which facilitates the formation of multi-hexamers. These multi-hexamers slowly release monomers, resulting in a prolonged and stable action profile.

Insulin is primarily administered by subcutaneous injections. The delivery of insulin via the ocular route has been challenging because of ocular barriers. These ocular barriers, while essential for the eye's protection and homeostasis, present significant challenges for delivering insulin via eye drops. Overcoming these barriers is crucial for the development of a viable ocular insulin delivery system.

Insulin is a large molecule that consists of 51 amino acids. It has a molecular weight of approximately 5.8 kD. Its relatively large size results in poor passive diffusion across the ocular barriers—the corneal epithelium, tight junctions between cells, active efflux transporters-significantly reducing its absorption and bioavailability when delivered via eye drops.

In addition, the lipophilic nature of the corneal epithelium poses a considerable barrier to the absorption of hydrophilic molecules. Insulin exhibits hydrophilic properties, which further impedes its passive diffusion.

In addition to the corneal barrier, the conjunctiva and sclera contribute to ocular drug absorption limitations. While these tissues are more permeable to hydrophilic molecules than the cornea, their ability to permit the passage of large molecules like insulin is still limited.

Ocular absorption is affected by precorneal factors. Eye drops have a short residence time due to rapid evacuation through tear drainage. Further, tear fluid contains peptidases and proteases that can potentially degrade insulin, reducing its bioavailability.

Currently, there are no insulin eye drops available to lower blood glucose levels. Insulin is typically administered through injections or an insulin pump, as it needs to be delivered directly into the bloodstream to be effective.

The present invention leverages a previously undescribed effect of prostaglandin and prostaglandin analogues on the permeability of the cornea and conjunctiva to large molecules for use as a penetration enhancer for insulin.

Prostaglandin and prostaglandin analogues have been used clinically as ocular hypotensives since 1998. The first prostaglandin analogue to be approved by the FDA for clinical ophthalmic use is latanoprost. Later drugs include Unoprostone, Travoprost, Bimatoprost, Tafluprost, Latanoprostene.

Prostaglandin analogues are prodrugs which are hydrolyzed in the cornea into their active form to selectively stimulate the prostaglandin F2 alpha receptor to cause up-regulation of matrix metalloproteinases and remodeling of the extracellular matrix in the structures of the eye adjacent to Schlemm's canal where aqueous humor drains. These actions result in higher tissue permeability of these structures, thereby decreasing outflow resistance to aqueous which results in a drop in intraocular pressure. Aqueous contains cells and large proteins.

Although no studies have been performed to show similar effects on the cornea, changes in biomechanical properties of the cornea after local prostaglandin analogue treatment suggest that similar changes occur in the cornea.

Treatment with latanoprost has been shown to increase corneal hysteresis not correlated with the drug-induced decrease in the intraocular pressure. This suggests a direct effect of latanoprost on the viscoelastic corneal properties^{[1]},^{[2]},^{[3]}.

Other prostaglandin analogues (travoprost, latanoprost and bimatoprost) have likewise been associated with different extents of reduction in tissue stiffness and changes in corneal microstructure [4]

The biomechanical effects of prostaglandin analogues on the cornea suggest that changes to corneal tissue permeability may also be occurring, although no studies have been performed to confirm this. The present invention leverages this effect to increase the ocular absorption of insulin.

### PRIOR ART

U.S. Pat. No. 10,335,418 ('418):

The '418 patent discloses a method and composition for treating metabolic syndrome, or a disorder associated with metabolic syndrome, e.g. obesity, dyslipidemia, and/or a diabetic condition. An F-series prostaglandin (i.e., latanoprost) according to the following formulas (I) and (II) are used:

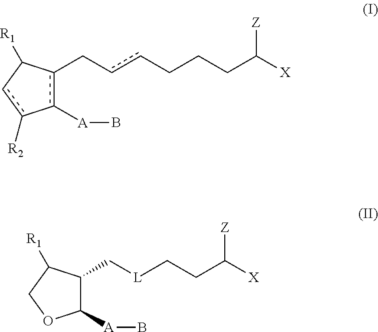

These include a compound of Formulas (I) or (II), or a pharmaceutically acceptable salt, hydrate, solvate, stereoisomer, polymorph, tautomer, isotopically enriched derivative, or prodrug thereof. The compounds are administered by any contemplated systemic route. A pharmaceutical composition of the formulas can be prepared, packaged, and/or sold in a formulation suitable for ophthalmic administration. Such formulations may, for example, be in the form of eye drops.

This reference fails to teach an insulin-latanoprost preparation.

International (WIPO) Patent Publication No. WO2010097800 ('800): The '800 reference teaches a non-hypoglycemic or euglycemic combination of insulin and prostaglandin analogues for injecting, implanting and/or inserting in/or adjacent to the Schlemm's canal or at other ocular tissues and sites related to or surrounding the Schlemm's canal, wherein the hypotensive peptides include insulin.

This reference fails to teach the use of prostaglandin analogues as a penetration enhancer to enhance ocular absorption of topically applied insulin. It also fails to teach a combination of insulin and prostaglandin analogues for topical ocular application to lower blood glucose levels. The disclosure specifically excludes hypoglycemic compositions by specifically limiting itself to euglycemic insulin.

### SUMMARY AND OBJECTIVE OF THE INVENTION

The primary objective of this invention is to:

Provide a method to increase the penetration of insulin when applied topically to the eye.

Utilize the penetration-enhancing properties of the prostaglandin analogue to facilitate the transport of insulin across the corneal and conjunctival barriers, resulting in improved bioavailability and therapeutic efficacy.

Provide a formulation comprising an insulin and a prostaglandin analogue that causes a significant systemic hypoglycemic effect in order to treat diabetes mellitus.

Provide a comfortable and patient-friendly alternative to subcutaneous insulin injections, reducing the burden of frequent injections and improving patient compliance.

Offer a versatile platform for ocular drug delivery, which can be further tailored and optimized for the administration of other therapeutic agents with limited ocular penetration.

The present invention is directed to a novel insulin delivery system that utilizes the ocular application of the insulin.

The present invention is directed to a novel insulin delivery system that provides at least an insulin, in a form of rapid- or short-acting type, and at least a prostaglandin analogue.

The present invention is directed to a novel insulin formulation that comprises at least an insulin, in a form of rapid- or short-acting type, and at least a prostaglandin analogue.

The present invention is directed to an insulin with a prostaglandin analogue formulation further comprising of excipients used in ocular application.

The present invention is directed to a process of manufacturing an ocular application solution with insulin and prostaglandin analogue.

This invention has the potential to revolutionize the management of diabetes by offering a more convenient and efficient method of insulin administration, while minimizing the complications and challenges associated with traditional subcutaneous injections.

### DETAILED DESCRIPTION OF THE INVENTION

The present invention will be further disclosed in the following paragraphs.

The terminologies used in the disclosure will be considered as used in the field of technology of the present invention. No new usage of these words are being introduced into the further disclosure.

As already presented in the previous sections, the present invention is directed to a novel ocular insulin delivery system that utilizes insulin together with a prostaglandin analogue.

The present invention utilizes insulin identified as rapid-acting or short-acting insulin.

The present invention utilizes rapid-acting insulin such as but not limited to: Insulin lispro, and Insulin aspart.

The present invention utilizes short-acting insulin such as but not limited to Regulalr insulin (insulin R).

The present invention utilizes penetration enhancers.

The present invention utilizes penetration enhancer such as prostaglandin analogues.

The present invention utilizes prostaglandin analogues such as but not limited to the following: latanoprost, bimatoprost, travoprost, or tafluprost.

The present invention leverages the corneal penetration-enhancing properties of prostaglandin analogues to facilitate the transport of insulin across the corneal and conjunctival barriers, resulting in a significant systemic hypoglycemic effect.

The present invention utilizes the excipients and other ingredients which are commonly used in the preparation of an ocular medication such as, but not limited to:
Preservatives: benzalkonium chloride (final concentration range can be from 0.005% to 0.08%)
Surfactants: polysorbate 80, polyoxyl 40 hydrogenated castor oil, metacresol
Chelating agents: disodium edetate, tromethamine
Buffering agents: sodium phosphate dibasic, sodium phosphate monobasic, citric acid monohydrate, sodium hydrogen phosphate dihydrate, hydrochloric acid, sodium hydroxide, boric acid, sodium borate
Viscosity enhancers: HPMC 4000 cps, PVA, Carbomer, PEG, Na hyaluronate, glycerin
Tonicity agents: sodium chloride, glycerol, zinc chloride, zinc oxide
pH adjusting agents: HCl/NaOH q.s. to pH 6.4 to 7.4
Solvent: water for injection

The concentration of insulin in the present invention ranges from 1.735 mg/mL to 69.4 mg/mL or a percentage of 0.1735% to 6.94%.

The concentration of prostaglandin analogue in the present invention ranges are different depending on which analogue will be used.

For Latanaprost, the final concentration range is from 0.000625% to 0.2%.

For Unoprostone, the final concentration range is from 0.015% to 0.6%.

For Travoprost, the final concentration range can be from 0.002% to 0.16%.

For Bimatoprost, the final concentration range can be from 0.001% to 0.12%.

For Tafluprost, the final concentration range can be from 0.00001875% to 0.06%. Prostaglandin analogues are relative insoluble and must be dissolved together with a surfactant at a high temperature typically at 80° C. then cooled down before mixing with insulin because insulin denatures above 40° C.

Insulin itself is relatively insoluble at pH 6.4 to 7.4 and must be dissolved in water at pH 2-3. The pH is then adjusted through buffering until the desired pH of 6.4 to 7.4 is achieved.

The present invention is prepared by mixing a solution of the selected insulin in water at the appropriate concentration with its pH adjusted to 6.4 to 7.4, with a solution of the selected prostaglandin analogue, likewise dissolved in water together with the selected surfactant at the appropriate concentration with its pH similarly adjusted. The selected excipients are added to the combination while maintaining the pH through buffering. The mixture is gently agitated until the components are thoroughly combined, resulting in a homogeneous insulin eye drop solution.

The present invention is administered topically onto the eye depending on the amount as recommended by the user's health care professional.

As established in the background of the present invention, there are different factors that hinder the application of insulin via the ocular route. The most prominent factors are the biological factors and how insulin interacts with these.

As a relatively large molecule, insulin poses a significant challenge for ocular absorption. Insulin also has 18 hydrophilic residues on its surface which renders it difficult to be absorbed through the lipophilic barriers of the eye. Tear turnover through the nasolacrimal drainage also reduces drug contact time with the cornea thus limiting absorption.

The technical solution being presented by the present invention which was not disclosed by the prior art is the use of prostaglandin analogues as penetration enhancers for insulin and the combination of prostaglandin analogue with insulin for the treatment of diabetes mellitus. It was not known previously that this combination of drugs is possible for the treatment of diabetes mellitus through topical ocular application.

As found in the prior arts, prostaglandin analogues are hydrolyzed in the cornea into their active form to selectively stimulate the prostaglandin F2 alpha receptor to cause up-regulation of matrix metalloproteinases and remodeling of the extracellular matrix in the structures of the eye adjacent to Schlemm's canal where aqueous humor drains. These actions result in higher tissue permeability of these structures, thereby decreasing outflow resistance to aqueous which results in a drop in intraocular pressure.

This mechanism also allows the increased uptake of insulin that is applied topically onto the eye, thus resolving the problems of the prior arts.

To further increase the uptake of insulin, excipients such as polysorbate 80 and benzalkonium chloride are being used by the present invention. These excipients affect the corneal epithelial barrier by allowing increased paracellular transport.

This novel administration of the insulin formulation also allows increased compliance by users since it removes the fear of needles which is a very invasive method of introducing insulin into the bloodstream.

The present invention is further discussed in the following specific examples which are not aimed to limit the scope but instead gives embodiments on which the present invention were used and tested.

### EXAMPLES

### EXAMPLE 1. Formulation of Insulin Eye Drop using Insulin regular and

| | |
|---|---|
| Insulin regular | 0.1735% |
| Latanoprost | 0.0025% |
| Benzalkonium Chloride | 0.01% |
| Polysorbate 80 | 0.1% |
| Buffering agent | q.s buffer capacity nmt 0.5% |
| Tonicity agent | 0.5-2% NaCl *quantity sufficient to osmolality |
| approx. 308 | mOsMol/Kg* |
| Solvent | q.s. 2.5 mL |

### EXAMPLE 2. Formulation of Insulin Eye Drop using Insulin lispro and

| | |
|---|---|
| Insulin lispro | 0.1735% |
| Tafluprost | 0.0025% |
| Polysorbate 80 | 0.5% |
| Buffering agent | q.s buffer capacity nmt 0.5% |
| Tonicity agent | 0.5-2% NaCl *quantity sufficient to osmolality |
| approx. 308 | mOsMol/Kg* |
| Solvent | q.s. 2.5 mL |

### EXAMPLE 3. Formulation of Insulin Eye Drop using Insulin aspart and

| | |
|---|---|
| Insulin aspart | 6.94% |
| Bimatoprost | 0.12% |
| Benzalkonium Chloride | 0.01% |
| Polysorbate 80 | 0.5% |
| Buffering agent | q.s buffer capacity nmt 0.5% |
| Tonicity agent | 0.5-2% NaCl *quantity sufficient to osmolality |
| approx. 308 | mOsMol/Kg* |
| Solvent | q.s. 2.5 mL |

### EXAMPLE 4. Formulation of Insulin Eye Drop using Insulin regular and

| | |
|---|---|
| Insulin regular | 0.1735% |
| Travoprost | 0.002% |
| Polysorbate 80 | 0.5% |
| Buffering agent | q.s buffer capacity nmt 0.5% |
| Tonicity agent | 0.5-2% NaCl *quantity sufficient to osmolality |
| approx. 308 | mOsMol/Kg* |
| Solvent | q.s. 2.5 mL |

### EXAMPLE 5. Formulation of Insulin Eye Drop using Insulin regular and

| | |
|---|---|
| Insulin regular | 0.1735% |
| Unoprostone | 0.015% |
| Benzalkonium chloride | 0.01% |
| Polysorbate 80 | 0.5% |
| Buffering agent | q.s buffer capacity |
| Tonicity agent | 0.5-2% NaCl *quantity sufficient to osmolality |
| approx. 308 | mOsMol/Kg* |
| Solvent | q.s. 2.5 mL |

EXAMPLE 6. Insulin regular with Latanoprost dose response

Fasting interstitial glucose was measured (using an Abbott Freestyle Libre Continuous Glucose Monitor). The test drug (0.1735% insulin r vs 0.1735 insulin r+0.0025% latanoprost) was instilled in both eyes thereafter. Interstitial glucose was then measured serially for 8 hours. Fasting levels-trough levels and time to trough in hours were compared relative to the fasting levels.

| | | 0.02 mL | 0.02 mL | 0.05 mL | 0.10 mL |
|---|---|---|---|---|---|
| | 0.02 mL | insulin r + | insulin r + | insulin r + | insulin r + |
| | insulin r | latanoprost | latanoprost | latanoprost | latanoprost |
| | only | @ eye | @ eye | @ eye | @ eye |
| Fasting mg/dL | 159 | 144 | 132 | 146 | 141 |
| Trough mg/dL | 152 | 115 | 100 | 100 | 67 |
| Fasting − trough mg/dL | 7 | 29 | 32 | 46 | 74 |
| Time to trough hrs | n/a | 5 | 2 | 6.5 | 5 |

EXAMPLE 7. Insulin lispro with Tafluprost (no BAK)

Fasting interstitial glucose was measured (using an Abbott Freestyle Libre Continuous Glucose Monitor). The test drug (0.1735% insulin lispro vs 0.1735% insulin lispro+0.0025% latanoprost) was instilled in both eyes thereafter. Interstitial glucose was then measured serially for 3 hours. Levels at 3 hours were compared relative to the fasting levels.

| | 0.02 mL | 0.02 mL lispro + | 0.05 mL lispro + |
|---|---|---|---|
| | lispro @eye | tafluprost @eye | tafluprost @eye |
| Fasting mg/dL | 112 | 162 | 144 |
| 3 hours mg/dL | 108 | 129 | 91 |
| Difference | 4 | 33 | 53 |

### CERTAIN EMBODIMENTS

In certain embodiments, the disclosed compositions and methods further relate to sub-therapeutic or non-IOP-lowering concentrations of prostaglandin analogues configured to enhance corneal and scleral permeability for macromolecular drug delivery. The following description provides additional exemplary details of such embodiments.

The non-therapeutic dose for each prostaglandin analogue can be defined as a concentration lower than the minimum effective dose that produces a therapeutic reduction in intraocular pressure (IOP). For example, the clinically used concentrations for latanoprost, bimatoprost, travoprost, tafluprost, and unoprostone are known to lower IOP by several millimeters of mercury. Sub-therapeutic concentrations, those at least one order of magnitude below these levels, would therefore not measurably or therapeutically alter IOP but could still activate prostaglandin FP receptors in ocular tissues. These receptors are distributed in the ciliary muscle, sclera, corneal epithelium, and trabecular meshwork. Activation at sub-threshold levels can induce mild upregulation of matrix metalloproteinases (MMP-1, MMP-2, MMP-9) and subtle remodeling of extracellular matrix (ECM) components such as collagen and proteoglycans without altering aqueous humor outflow or ocular pressure. This receptor-mediated matrix modification can increase paracellular and transcellular permeability across corneal and scleral tissues. Although the IOP remains unaltered, the loosening of collagen networks and reduction of tight junction protein expression (e.g., occludin, claudin-5, ZO-1) facilitates the diffusion of large peptides such as insulin through the ocular surface and into anterior segment tissues. Therefore, even at non-therapeutic doses, prostaglandin analogues can create transient, reversible microstructural changes that promote macromolecule transport. This provides a mechanistic explanation for enhanced insulin absorption independent of pressure modulation.

The advantage of achieving increased ocular permeability without altering IOP is clinically significant. Patients with normal or low IOP, particularly those predisposed to ocular hypotony, cannot safely tolerate additional reductions in pressure. Prolonged hypotony (IOP below 6 mmHg) can lead to retinal folding, choroidal effusion, and vision impairment. A formulation that selectively enhances absorption without affecting IOP avoids these complications, making it safer for broader use, including individuals with normal-tension glaucoma, post-surgical eyes, or ocular trauma. Furthermore, by separating permeability modulation from pressure regulation, the invention introduces a targeted therapeutic mechanism applicable to ocular drug delivery rather than glaucoma treatment. This separation distinguishes the invention from prior art that relies exclusively on IOP-lowering effects for utility.

A representative formulation for each prostaglandin analogue may include sub-therapeutic concentrations. Example formulations include: for latanoprost, 0.00025% (w/v) latanoprost and 10 mg/mL recombinant human insulin in isotonic phosphate buffer with 0.05% polysorbate 80, 0.3% hydroxypropyl methylcellulose, and 0.005% benzalkonium chloride; for bimatoprost, 0.005% (w/v) bimatoprost with 8-10 mg/mL insulin in a similar excipient system; for travoprost, 0.0005% (w/v) travoprost with 10 mg/mL insulin; for tafluprost, 0.00001875% (w/v) tafluprost with 10 mg/mL insulin; and for unoprostone, 0.02% (w/v) unoprostone with 10 mg/mL insulin. Each formulation uses physiologically acceptable buffers, tonicity agents, viscosity enhancers, and preservatives suitable for ophthalmic administration. These compositions may be prepared as sterile aqueous solutions stored in light-protected containers at neutral pH.

In an alternative embodiment, the prostaglandin analogue and insulin are administered sequentially rather than as a single formulation. The method includes instilling one drop of a prostaglandin analogue solution at the specified sub-therapeutic concentration into the eye and allowing a short latency period, typically 10 to 15 minutes, to permit partial activation of FP receptors and localized extracellular matrix remodeling. Following this preconditioning interval, a second eye drop containing the insulin formulation is applied to the same eye. This sequential administration maximizes permeability enhancement while preventing potential degradation or incompatibility between prostaglandin and peptide components within a single container. The same method can be implemented as part of a pharmaceutical kit comprising two separate compositions and printed instructions for sequential use. This dual-step approach allows flexible dosing and ensures that the prostaglandin acts primarily as a temporary permeability modulator without continuous pharmacologic interaction with insulin. By enabling macromolecule penetration without altering intraocular pressure, the described composition and method provide a novel, non-obvious, and physiologically safe platform for topical ocular delivery of peptide therapeutics.

For latanoprostene bunod, the final concentration range can be from about 0.0024% to 0.096% (w/v). In some embodiments, the latanoprostene bunod may be dissolved in an aqueous solution containing one or more surfactants, such as polysorbate 80 or polyoxyl castor oil, to facilitate solubility prior to blending with the insulin solution. The pH of the latanoprostene bunod solution may be adjusted to approximately pH 6.4 to 7.4 prior to mixing with the insulin component. Equivalent ophthalmically acceptable buffers, surfactants, and solubilizing systems may be used without departing from the scope of the invention.

As used herein, the terms “comprise,” “comprising,” “include,” and “including” are intended to be open-ended and mean “including but not limited to.” The singular forms “a,” “an,” and “the” include plural referents unless the context clearly dictates otherwise. “Or” is generally employed in its non-exclusive sense (i.e., “and/or”).

Unless otherwise indicated, numerical ranges include all values and sub-ranges therein, and endpoints are included. A value modified by “about” encompasses ±10% of the recited value or such lesser variation as would be understood by a person of ordinary skill in the art for the particular context.

Examples, embodiments, and data described herein are provided for illustration and are not intended to limit the scope of the claims. Features described in connection with any embodiment may be combined with features of any other embodiment unless such combination is technically incompatible.

Where a range, list, or set of alternatives is provided (e.g., types of insulin, prostaglandin analogues, excipients, concentrations, dosage regimens), any individual member or sub-combination thereof may be claimed as if individually recited, and any suitable equivalents may be substituted.

Discussions of putative mechanisms (e.g., receptor engagement, MMP upregulation, ECM remodeling, tight-junction modulation) are provided to aid understanding and do not limit the claimed subject matter to any particular mechanism of action unless expressly recited in a claim.

The disclosure contemplates kits comprising one or more containers holding the described compositions (e.g., insulin formulation and prostaglandin analogue formulation), optionally with printed instructions for sequential administration. Methods may be performed in any order consistent with the intended result unless a specific order is expressly required.

The disclosure encompasses obvious modifications and functional equivalents of the components, concentrations, excipients, and procedures disclosed herein, as would be recognized by a person of ordinary skill in the art in view of this specification.

The identification of any document or subject matter as “prior art” is not an admission that such document or subject matter constitutes prior art under any statute, rule, or case law.

What is claimed is:

1. A pharmaceutical composition comprising: (a) an insulin; (b) a prostaglandin analogue; and (c) one or more pharmaceutically acceptable excipients; wherein the composition is formulated for topical ophthalmic administration.

2. The pharmaceutical composition of claim 1, wherein the insulin comprises a rapid-acting insulin or a short-acting insulin.

3. The pharmaceutical composition of claim 2, wherein the rapid-acting insulin is selected from the group consisting of insulin lispro, insulin aspart, and insulin glulisine.

4. The pharmaceutical composition of claim 2, wherein the short-acting insulin comprises regular insulin.

5. The pharmaceutical composition of claim 1, wherein the prostaglandin analogue is selected from the group consisting of latanoprost, unoprostone, bimatoprost, travoprost, tafluprost, and latanoprostene bunod.

6. The pharmaceutical composition of claim 1, wherein the insulin is present at a concentration from about 0.1735% to about 3.47% (w/v).

7. The pharmaceutical composition of claim 1, wherein the prostaglandin analogue is present at a sub-therapeutic concentration that does not reduce intraocular pressure by more than 3 mmHg within 24 hours after topical administration.

8. The pharmaceutical composition of claim 1, wherein the prostaglandin analogue comprises latanoprost at a concentration from about 0.0005% to about 0.1% (w/v).

9. The pharmaceutical composition of claim 1, wherein the prostaglandin analogue comprises unoprostone at a concentration from about 0.015% to about 0.6% (w/v).

10. The pharmaceutical composition of claim 1, wherein the prostaglandin analogue comprises travoprost at a concentration from about 0.004% to about 0.16% (w/v).

11. The pharmaceutical composition of claim 1, wherein the prostaglandin analogue comprises bimatoprost at a concentration from about 0.001% to about 0.12% (w/v).

12. The pharmaceutical composition of claim 1, wherein the prostaglandin analogue comprises tafluprost at a concentration from about 0.00001875% to about 0.06% (w/v).

13. The pharmaceutical composition of claim 1, wherein the prostaglandin analogue comprises latanoprostene bunod at a concentration from about 0.0024% to about 0.096% (w/v).

14. A method of enhancing ocular absorption of insulin in a subject, the method comprising topically administering to an eye of the subject a composition comprising: (i) an insulin; and (ii) a prostaglandin analogue; wherein the prostaglandin analogue increases corneal permeability sufficient to permit transcorneal transport of the insulin into ocular tissues or systemic circulation.

15. The method of claim 14, wherein the prostaglandin analogue is present at a concentration below its therapeutic intraocular-pressure-lowering range.

16. The method of claim 14, wherein the prostaglandin analogue is selected from the group consisting of latanoprost, bimatoprost, travoprost, tafluprost, unoprostone, and latanoprostene bunod.

17. The method of claim 14, wherein the insulin comprises a rapid-acting insulin or a short-acting insulin.

18. The method of claim 17, wherein the insulin is selected from the group consisting of insulin lispro, insulin aspart, insulin glulisine, and regular insulin.

19. The method of claim 14, wherein topical administration results in a reduction in ocular or interstitial glucose.

20. The method of claim 14, wherein topical administration results in a reduction in systemic glucose.
